(19) United States
(12) Patent Application Publication
(10) Pub. No.: US 2003/0008857 A1
(43) Pub. Date: Jan. 9, 2003

(54) Photodynamic therapy (PDT) in combination with factors that bind cell surface apoptosis-inducing receptors

(76) Inventors: David W.C. Hunt, White Rock (CA); Christopher M. Carthy, Vancouver (CA); David Granville, Coquitlam (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/193,003

(22) Filed: Jul. 10, 2002

Related U.S. Application Data
(63) Continuation of application No. 09/513,222, filed on Feb. 25, 2000.
(60) Provisional application No. 60/121,770, filed on Feb. 26, 1999.

(51) Int. Cl. A61K 31/555; A61K 31/409; A61N 1/30

(52) U.S. Cl. 514/185; 514/410; 604/20

(57) ABSTRACT

The invention relates to photodynamic therapy (PDT) is used in combination with apoptosis-inducing agents to destroy target cells and tissues. Benefits of such combinations include 1) the ability to use lower doses of photosensitizers and/or light; 2) the ability to use lower doses of apoptosis-inducing agents; and 3) the ability to use apoptosis-inducing agents against target tissues which are otherwise insensitive to the apoptosis-inducing agents.

### RELATED APPLICATIONS

The present application claims the benefit of U.S. Provisional Application 60/121,770, filed Feb. 26, 1999, which is hereby incorporated by reference in its entirety, as if fully set forth.

### FIELD OF THE INVENTION

This invention relates to the use of photodynamic therapy (PDT) in combination with apoptosis-inducing agents, including the Fas ligand (FasL) and the tumor necrosis factor (TNF)-related apoptosis-inducing ligand (TRAIL), to destroy target cells. Target cells susceptible to such treatment include tumor cells, cells involved in intimal hyperplasia, virally-infected cells or autoimmune cells. The PDT may be conducted with any photosensitizer, and the combination permits the use of apoptosis-inducing agents which would otherwise be insufficiently specific for target cells of interest.

### BACKGROUND OF THE INVENTION

Photodynamic therapy (“PDT”) is an approved anti-cancer treatment that can be applied in many different circumstances, such as the treatment of superficial solid tumors, the removal of immunopathogenic cells such as those that related to psoriasis, the treatment of ocular neovascular disorders such as age-related macular degeneration, the removal atherosclerotic plaque and the prevention of restenosis. PDT involves the systemic or topical application of a light-absorbing photosensitizer, usually a porphyrin derivative, which accumulates somewhat selectively within target tissues. A highly potent photosensitizer is benzoporphyrin derivative monoacid ring A (“BPD-MA” or “verteporfin”), which is a second generation chlorin-type photosensitizer possessing distinct advantages over its hematoporphyrin forerunners in terms of effectiveness at low concentrations and its capacity to absorb activating light at longer and therefore more penetrating wavelengths of light. Upon irradiation with visible light of an activating wavelength, reactive oxygen species are produced in cells containing the photosensitizer, which directly leads to cell death. Evidence has been forwarded that PDT using different photosensitizers may cause cells to die through a distinctive process termed apoptosis. (Agarwal et al. Photodynamic therapy induces rapid cell death by apoptosis in L5178Y mouse lymphoma cells. Cancer Res. 51, 5993-5996 (1991); Luo et al. Rapid initiation of apoptosis by photodynamic therapy, Photochem. Photobiol. 63, 528-534 (1996); Granville et al., “Photodynamic therapy induces caspase-3 activation in HL-60 cells”, Cell Death and Different. 4, 623-628 (1997); Granville et al. “Overexpression of Bcl-XL prevents caspase-3-mediated activation of DNA fragmentation factor (DFF) produced by treatment with the photochemotherapeutic agent BPD-MA”, FEBS Lett. 422, 151-154 (1998); Granville et al, “Rapid cytochrome c release, activation of caspases 3, 6, 7 and 8 followed by Bap31 cleavage in HeLa cells treated with photodynamic therapy”, FEBS Lett. 437, 5-10 (1998)).

Apoptosis is the term used to describe a specific form of cell death that plays a critical role during normal development, differentiation, homeostasis or the normal cellular turnover within tissues. Apoptosis involves the activation within cells of a built-in program for cell suicide by which the cell essentially disassembles itself. This orderly form of cell death permits the cell to be processed into structures suitable for removal by phagocytic cells. Morphologically, apoptosis is characterized by the loss of contact with neighboring cells, surface membrane blebbing, condensation of the cytoplasm, endonuclease-mitigated chromatin condensation and segmentation of the nucleus. This organized disintegration of cells also includes the degradation of genomic DNA into regular nucleosomal fragments.

A suicide program is induced within cells bearing receptors for certain members of the tumor necrosis factor (TNF) family of molecules, such as Fas ligand (FasL), TNF-related apoptosis-inducing ligand (TRAIL, APO-2L), or TNF itself, when they bind to the specific cell surface receptors. (Suda et al., “Molecular cloning and expression of the Fas ligand, a novel member of the tumor necrosis factor family”, Cell, 75, 1169-1178 (1993); “Purification and characterization of the Fas-ligand that induces apoptosis”, J. Exp. Med., 179, 873-879 (1994); Pitti et al., “Induction of apoptosis by Apo-2 ligand, a new member of the tumor necrosis factor cytokine family”, J. Biol. Chem., 271, 12687-12690 (1996)). The receptors for FasL, TNF and TRAIL are members of a TNF receptor superfamily and include Fas (APO-1, CD95), TNF receptor-1 (TNFR-1) and at least 2 closely related receptors termed TRAIL receptor-1 (TR-1) or death receptor-4 (DR4) and TR-2 (DR5). (Itoh et al., “The polypeptide encoded by the cDNA for human cell surface antigen Fas can mediate apoptosis”, Cell, 66, 233-243 (1991); Oehm et al., “Purification and molecular cloning of the APO-1 cell surface antigen, a member of the tumor necrosis factor/nerve growth factor receptor superfamily: sequence identity with Fas antigen”, J. Biol. Chem., 267, 10709-10715 (1992); Nagata, S., “Apoptosis by death factor”, Cell, 88, 355-365 (1997); Nagata and Goldstein, “The Fas death factor” Science, 267, 1449-1456 (1995); Tartaglia et al, :“A novel domain within the 55 kd TNF receptor signals cell death”, Cell, 74, 845-853; Pan et al. “The receptor for the cytotoxic ligand TRAIL”, Science, 276, 111-113.; Pan et al., “An antagonist decoy receptor and a death domain-containing receptor for TRAIL”, Science, 277, 815-818 (1997); Sheridan et al. “Control of TRAIL-induced apoptosis by a family of signaling and decoy receptors”, Science, 277, 818-821 (1997); Ashkenazi and Dixit, “Death receptors: Signaling and modulation”, Science, 281, 1305-1308 (1998)).

Apoptosis may be triggered by the binding of the Fas receptor by its natural ligand (FasL) or agonistic anti-Fas antibodies. (Suda et al., “Molecular cloning and expression of the Fas ligand, a member of the tumor necrosis factor family”, Cell. 75, 1169-178 (1993)). Fas is expressed by many different cell types and its presence signifies that these cells may be receptive to apoptosis-inducing signals from FasL-bearing cells. In the periphery, Fas-FasL interactions serve to limit the proliferation of activated T cells, promote the lysis of virally-infected cells by cytotoxic T cells, and contribute to the maintenance of a state of immune privilege in different tissues by imperiling the survival of activated inflammatory cells. (Nagata, S., “Apoptosis by death factor”, Cell 88, 355-365 (1997)). Normal and malignant cells may express Fas and/or FasL. Administration of anti-Fas antibodies to mice may cause widespread tissue effects and may lead to the death of these animals from liver damage. (Ogasawara et al. “Lethal effect of the anti-Fas antibody in mice”, Nature, 364, 806-809 (1993); Rodriguez et al., “Systemic injection of a tripeptide inhibits the intracellular activation of CPP332-like proteases in vivo and fully protects mice against Fas-mediated fulminant liver destruction and death”, J. Exp. Med., 184, 2067-2072 (1996)).

TNF may induce apoptosis in a wide range of cell types bearing the TNFR-1. However, cells may be protected form the lethal effects of TNF through the activation of the transcription factor nuclear kappa B (NF-κB) that promotes the transcription of genes encoding various a number of anti-apoptotic factors. (Wang et al. “TNF- and cancer therapy-induced apoptosis: potentiation by inhibition of NF-κB”, Science, 274, 784-787 (1996); Antwerp et al., “Suppression of TNF-α-induced apoptosis by NF-κB”, Science, 274, 787-789).

TRAIL and its receptors are widely expressed in many human tissues. Importantly, TRAIL rapidly activates apoptosis in many transformed cell lines but not in normal cell types, even though both forms express DR4 and DR5. It is believed that normal cells are provided a degree of protection from TRAIL-mediated apoptosis through their expression of “decoy” cell surface receptors (termed decoy receptor 1, DcR1 or TRID) that bind TRAIL but do not transduce a signal to the cell and therefore do not induce apoptosis in normal cells. Transformed cells also to express the decoy receptors for TRAIL. (Pan et al. “The receptor for the cytotoxic ligand TRAIL”, Science, 276, 111-113.; Pan et al., “An antagonist decoy receptor and a death domain-containing receptor for TRAIL”, Science, 277, 815-818 (1997); Sheridan et al. “Control of TRAIL-induced apoptosis by a family of signaling and decoy receptors”, Science, 277, 818-821 (1997)). Relative levels of decoy and death domain-containing TRAIL receptors may also influce cell sensitivity to trail.

Intracellular regulators of apoptosis may also influence cell sensitivity to TRAIL. Human melanoma cell sensitivity to TRAIL increased with decreases in levels of the FLICE-inhibitory protein (FLIP), a molecule known to interact with and regulate the sensitivity of the Fas signaling pathway (Griffith et al. “Intracellular regulation of TRAIL-induced apoptosis in human melanoma cells.” J. Immunol. (1998) 161:2833-2840).

TRAIL may play a role in the normal immune system or the action of immune cells against virally-infected cells. T cells from patients infected with the human immunodeficiency virus (HIV) exhibit sensitivity to TRAIL-mediated killing. (Jeremias et al., “TRAIL/Apo-2-ligand-induced apoptosis in human T cells”, Eur. J Immunol. 28, 143-152 (1998)).

Apoptosis may ensue upon the binding FasL, TNF or TRAIL to their specific cell surface receptors. Although the details of these events are not as well understood for the TRAIL receptors DR4 and DR5, FasL and TNF cause the recruitment of specific adapter proteins to the intracellular N-terminal domains of Fas and TNFR-1, respectively. These linking molecules have been termed Fas-associated death domain (FADD) and TNFR-associated death domain (TRADD). (Boldin et al., “Involvement of MACH, a novel MORT1/FADD-interacting protease, in Fas/APO-1- and TNF receptor-induced cell death”, Cell, 85, 803-815 (1996); Muzio et al., “FLICE, a novel FADD-homologous ICE/CED-3-like protease, is recruited to the CD95 (FAS/APO-1) death-inducing signaling complex”, Cell, 85, 817-827 (1996)). FADD and TRADD recruit the death protease caspase-8 an event that leads to the processing and activation of caspase-8. Activated caspase-8 mobilizes a cascade of caspase activity involving other caspase family members and leading to the degradation of specific cell proteins, DNA fragmentation and the characteristic morphologic changes associated with apoptosis. (Hirata et al, “Caspases are activated in a branched cascade and control distinct downstream processes in Fas-induced apoptosis”, J. Exp. Med., 187, 587-600).

Human myeloid leukemia HL-60 cells treated with verteporfin and light contained activated caspase-3, a member of the family of cysteine death proteases, leading to the degradation of specific intracellular proteins, DNA fragmentation and apoptotic cell death. (Granville et al., “Photodynamic therapy induces caspase-3 activation in HL-60 cells”, Cell Death Differ. 4, 623-628 (1997); Granville et al., “Overexpression of Bcl-XL prevents caspase-3-mediated activation of DNA fragmentation (DFF) produced by treatment with the photochemotherapeutic agent BPD-MA”, FEBS Letters. 422, 151-154 (1998)). HeLa cells treated with verteporfin and light exhibited morphologic signs of apoptosis and evidence of the activation of caspases-3, 6, 7 and 8 (FLICE/MACH/Mch5). (Granville et al., “Rapid cytochrome c release, activation of caspases 3, 6, 7 and 8 followed by Bap31 cleavage in HeLa cells treated with photodynamic therapy”, FEBS Lett. 437, 5-10 (1998)). Fas receptor ligation and subsequent receptor chain trimerization mobilizes caspase-8 which in turn processes caspase-3, leading to the full activation of the protease cascade. (Boldin et al., “Involvement of MACH, a novel MORT1/FADD-interacting protease, in Fas/APO-1- and TNF receptor-induced cell death”, Cell. 85, 803-815 (1996); Muzio et al, “FLICE, a novel FADD-homologous ICE/CED-3-like protease, is recruited to the CD95(Fas/APO-1) death-inducing signaling complex”, Cell 85, 817-827, (1996); Hirata et al., “Caspases are activated in a branched protease cascade and control downstream processes in Fas-induced apoptosis”, J. Exp. Med. 187, 587-600 (1998)). Caspase-3 activity during apoptosis is directly linked to DNA fragmentation. (Casciola-Rosen et al., “Apopain/CPP32 cleaves proteins that are essential for cellular repair: a fundamental principle of apoptotic death”, J. Exp. Med. 183, 1957-1964 (1996); Janicke et al., “Caspase-3 is required for DNA fragmentation and morphological changes associated with apoptosis”, J. Biol. Chem. 273, 9357-9360 (1998)). Since PDT with verteporfin as well as Fas receptor ligation cause caspase-3 activation and DNA fragmentation, certain biochemical events triggered by these agents may be similar.

It has been indicated that different chemotherapeutic drugs such as doxorubicin (adriamycin) may augment the effect of anti-Fas antibody upon the induction of apoptosis in human tumor cell lines. (Nakamura et al., “Induction of apoptosis in HL60 cells by anticancer drugs in combination with anti-Fas monoclonal antibody”, Anticancer Res. 17, 173-180 (1997); Mizutani et al., “Doxorubicin sensitizes human bladder carcinoma cells to Fas-mediated cytotoxicity”, Cancer, 79, 1180-1189 (1997); McGahon et al., “Chemotherapeutic drug-induced apoptosis in human leukaemic cells is independent of the Fas (APO-1/CD95) receptor ligand system”, Br. J. Haematol., 101, 539-47 (1998)). One study has evaluated the effect of chemotherapeutic agents combined with TRAIL on apoptosis in breast cell lines. Doxorubicin and 5-fluorouracil enhanced the effect of TRAIL in the induction of apoptosis and the combined effects were mediated through caspase activation. Agents that did not act in combination with TRAIL independently produced minimal caspase activation. (Keane et al. “Chemotherapy augments TRAIL-induced apoptosis in breast cell lines”, Cancer Res., 59, 734-741 (1999)).

The capacity of different cytotoxic drugs to act in concert with PDT upon tumor cell killing has been studied. (Varnes et al., “Enhancement of photodynamic killing (with chloroaluminum phthalocyanine) by treatment of V79 cells with the lonophore nigericin”, Cancer Res, 50, 1620-1625 (1990); Baas et al., “Enhancement of photodynamic therapy by mitomycin C: a preclinical and clinical study”, Br. J. Cancer, 73, 945-951 (1996); Gantchev et al. “Combination toxicity of etoposide (VP-16) and photosensitization with a water-soluble aluminum phthalocyanine in K562 human leukaemic cells”, Br. J. Cancer, 74, 1570-1577 (1996); Gantchev and Hunting, “Enhancement of etoposide (VP-16) cytotoxicity by enzymatic and photodynamically induced oxidative stress”, Anticancer Drugs, 8, 164-173 (1997); Casas et al., “Enhancement of aminolevulinic acid based photodynamic therapy by adriamycin”, Cancer Lett., 121, 105-113 (1997)). No mechanisms that account for the capacity of different drugs to enhance the effect of PDT have been defined.

Although TRAIL and FasL have been shown to have anti-tumor effects, both appear to have limitations for therapeutic purposes. As noted by Keane et al. “Chemotherapy augments TRAIL-induced apoptosis in breast cell lines”, Cancer Res., 59, 734-741 (1999), some types of tumor cells are resistant to TRAIL. The application of FasL in effective amounts in clinical situations may be problematic since many normal cells express Fas and antibodies known to trigger the Fas receptor have been shown to cause severe tissue damage and death when given to mice. (Ogasawara et al. “Lethal effect of the anti-Fas antibody in mice”, Nature, 364, 806-809 (1993); Rodriguez et al., “Systemic injection of a tripeptide inhibits the intracellular activation of CPP332-like proteases in vivo and fully protects mice against Fas-mediated fulminant liver destruction and death”, J. Exp. Med, 184, 2067-2072 (1996)). In order to devise a treatment using these apoptosis-inducing agents, it would be useful to have a method of augmenting their effects which is itself non-toxic.

### SUMMARY OF THE INVENTION

The invention is directed to the use of photodynamic therapy (PDT) in combination with apoptosis-inducing agents which bind to receptors on the surface of target cells. This results in the induction of apoptosis leading to the destruction of the target cells. Generally, the invention encompasses the treatment of target cells with an apoptosis-inducing agent either before, during, or after PDT treatment, resulting in an enhancement of target cell destruction. Additional applications of the combination include inhibiting the proliferation of the target cells and inhibiting the growth of tissues comprising the target cells. The methods of the invention offer the benefit of using apoptosis-inducing agents which would otherwise be insufficiently specific for target cells of interest, either by deleterious effects on non-target cells or by lack of efficacy against some target cells of interest.

Accordingly, in one aspect, the invention is directed to a method of enhancing the destruction of target cells by apoptosis using photodynamic therapy (PDT) in combination with apoptosis-inducing agents, comprising the steps of:

(a) exposing the cells to a sufficient amount of a photosensitizer to permit an effective amount to localize in the target cells,

(b) permitting a sufficient time to elapse to allow an effective amount of the photosensitizer to localize in the target cells,

(c) irradiating the cells with light absorbed by the photosensitizer at a sufficient energy level, and

(d) exposing the cells to a sufficient amount of at least one apoptosis-inducing agent wherein step (d) is carried out either before or after any of steps (a), (b) or (c), resulting in the induction of apoptosis in the target cells.

The methods of the present invention may be practiced with any photosensitizer and any apoptosis-inducing agent, each of which may be delivered systemically or locally.

In a preferred embodiment, the invention relates to methods wherein the photosensitizer is a green porphyrin, irradiation is with light absorbed by the green porphyrin, and the apoptosis-inducing agent is TRAIL or FasL, or a combination thereof.

In other aspects, the invention relates to the induction of 1) apoptosis, 2) DNA fragmentation, and 3) caspase activity as well as processing, by the combined use of PDT and apoptosis-inducing agent.

Moreover, the invention relates to formulations or compositions comprising both a photosensitizer and an apoptosis-inducing agent, preferably for use in the methods of the invention. The present invention includes pharmaceutical compositions to treat target cells, enhance their destruction or inhibit their proliferation, or inhibit the growth of tissues comprising said target cells. Such compositions contain effective amounts of a photosensitizer in combination with at least one apoptosis-inducing agent and a pharmaceutically acceptable carrier or excipient. Compositions individually containing the photosensitizer and apoptosis-inducing agent(s) for use together as needed are also encompassed.

Furthermore, the invention relates to the rapid and immediate induction of cytochrome c released from mitochondria into the cellular cytosol of target cells by PDT.

### BRIEF DESCRIPTION OF DRAWINGS

The present invention will be more clearly understood by referring to the following drawings:

FIG. 1. DNA fragmentation levels were determined for human Jurkat T cells treated with PDT, FasL or TRAIL. For PDT, cells were incubated with different amounts of verteporfin for 60 min at 37° C. and then irradiated with 690 nm light (5 J/cm²) delivered from light emitting diodes (LED). Increasing amounts of recombinant FasL or TRAIL were added to cells within parallel cultures. Following these treatments, cells were returned to the incubator and the state of nuclear DNA was assessed by PI staining and flow cytometric analysis 24 hours later. The percentage of cells containing sub-diploid (<2N) levels of DNA is given. The specific effect of each treatment was determined by subtracting the value obtained for cells maintained in medium alone from each experimental value. Hypodiploid levels of DNA were present in 13.8±0.7% of untreated Jurkat cells.

FIG. 2. Influence of TRAIL or FasL in combination with PDT upon DNA fragmentation. Jurkat cells were treated with different amounts of verteporfin (0-2 ng/ml) and irradiated with light (5 J/cm²) from LED panels 60 minutes later. Immediately after PDT, recombinant human TRAIL (20 ng/ml) or FasL (7.5 ng/ml) were added to the cultures. Following these treatments, cells were returned to the incubator and the state of nuclear DNA was assessed by PI staining and flow cytometric analysis 24 hours later. The percentage of cells containing sub-diploid (<2N) levels of DNA is given.

FIG. 3. Influence of FasL in combination with PDT upon caspase cleavage activity levels. Jurkat cells were exposed to increasing amounts of verteporfin for 60 minutes and then light-irradiated (5 J/cm²). FasL (final concentration=7.5 ng/ml) or an equal volume of culture medium was then added. Three hours following irradiation cell extracts were prepared. Caspase activity was assessed by protease assays using fluorogenic peptides containing the target amino acid consensus sequence for (A) caspase-3, (B) caspase-6 or (C) caspase-8 for cells treated with PDT and medium (□) or PDT plus FasL (▪). Mean values with standard deviations for triplicate measurements are shown.

FIG. 4. Influence of FasL in combination with PDT upon caspase activation and processing, as well as cleavage of the caspase substrate poly(ADP-ribose) polymerase (PARP), was assessed. Jurkat cells were exposed to increasing amounts of verteporfin for 60 minutes and then light-irradiated (5 J/cm²). FasL (final concentration=7.5 ng/ml) or an equal volume of culture medium was then added. Three hours following irradiation cell extracts were prepared. The state of caspase-3, -6, -8 and -9 as well as PARP was assessed by Western immunoblot analyses. Except for anti-caspase-6, the antibodies used for these studies are reactive against epitopes present within the proforms of these caspases as well as their processed activated forms (as indicated by arrows). The anti-caspase-8 antibody recognizes the a and b isoforms of this caspase. The anti-PARP antibody labels an epitope present within the intact molecule as well as the p85 cleavage product. Proteins were detected using the enhanced chemiluminescent detection system and bands visualized by autoradiographic techniques.

FIG. 5. Influence of TRAIL in combination with PDT upon caspase cleavage activity levels. Jurkat cells were exposed to increasing amounts of verteporfin for 60 minutes and then light-irradiated (5 J/cm²). TRAIL (final concentration=20 ng/ml) or an equal volume of culture medium was added. Three hours following irradiation cell extracts were prepared. Caspase activity was assessed by protease assays using fluorogenic peptides containing the target amino acid consensus sequence for (A) caspase-3, (B) caspase-6 or (C) caspase-8 for cells treated with PDT and medium (□) or PDT plus TRAIL (▪). Mean values with standard deviations of triplicate measurements are shown.

FIG. 6. Influence of TRAIL in combination with PDT upon caspase activation and processing, as well as cleavage of the caspase substrate PARP, was assessed. Jurkat cells were exposed to different amounts of verteporfin for 60 minutes and then light-irradiated (5 J/cm²). TRAIL (final concentration=20 ng/ml) or an equal volume of culture medium was then added. Three hours following irradiation cell extracts were prepared. The state of caspases-3, 6, 8 and 9 as well as PARP was assessed by Western immunoblot analyses. Except for anti-caspase-6, the antibodies used for these studies react against epitopes present within the caspase proforms as well as their processed activated forms (as indicated by arrows). The anti-caspase-8 antibody recognizes the a and b isoforms of this caspase. The anti-PARP antibody binds ah epitope present within the intact molecule and the p85 cleavage product. Proteins were detected using the enhanced chemiluminescent detection system and bands visualized by autoradiographic techniques.

FIG. 7. Influence of TRAIL and/or FasL in combination with PDT upon caspase cleavage activity levels. Jurkat cells were incubated verteporfin (5 ng/ml) for 60 minutes and then light-irradiated (5 J/cm²). FasL (final concentration=7.5 ng/ml) and/or TRAIL (final concentration=20 ng/ml) or an equal volume of culture medium was added. Three hours following irradiation cell extracts were prepared. Caspase activity was assessed by protease assays using fluorogenic peptides containing the target amino acid consensus sequence for caspase-3 (DEVD), caspase-6 (VEID) or caspase-8 (IETD). Mean values with standard deviations for triplicate measurements are shown.

FIG. 8. FasL and/or TRAIL combined with PDT leads to more extensive levels of caspase processing and PARP degradation. Jurkat cells were exposed to verteporfin (5 ng/ml) for 60 minutes and then light-irradiated (5 J/cm²). TRAIL (final concentration=20 ng/ml) and/or FasL (final concentration=7.5 ng/ml) or equal volumes of culture medium were then added. Three hours following irradiation, cell extracts were prepared. The state of caspase-3, -8 and -9 as well as PARP was assessed by Western immunoblot analyses. The antibodies utilized for these studies react against epitopes present within the proforms of these caspases as well as their processed activated forms (as indicated by arrows). Treatments are given within the figure.

FIG. 9. PDT with veteporfin elicits the immediate appearance of cytochrome c within the cytosol. Jurkat cells were incubated with increasing amounts of verteporfin for 60 minutes and then light-irradiated (5 J/cm²). Cytosolic extracts were prepared immediately afterward or 3 hours later. For this three hour period, TRAIL (final concentration=20 ng/ml) or FasL (final concentration=7.5 ng/ml) or an equal volume of medium was added. Cells extracts were separated by SDS-PAGE and transferred to nitrocellulose. These membranes were probed with an anti-cytochrome c antibody. Proteins were detected using the enhanced chemiluminescent detection system and bands visualized by autoradiographic techniques.

FIG. 10. The capacity of FasL and/or TRAIL to augment DNA fragmentation at increasing times following PDT was tested. Jurkat cells were incubated with verteporfin (2 ng/ml) for 60 minutes and then light-irradiated (5 J/cm²). TRAIL (final concentration=20 ng/ml) and/or FasL (final concentration=7.5 ng/ml) or equal volumes of culture medium were added 5 minutes after PDT or 1, 2 or 3 hours later. The state of nuclear DNA was assessed by PI staining and flow cytometric analysis 24 hours after PDT. The percentage of cells containing sub-diploid (<2N) levels of DNA is given. The specific effect of each treatment was determined by subtracting the value obtained for cells maintained in medium alone from each experimental value. Hypodiploid levels of DNA were present in 7.8±1.6, 9.5±0.7, 7.7±0.6 and 9.0±3.4% of control cells for the 5 minute, 1 hour, two hour and three hour factor addition times, respectively.

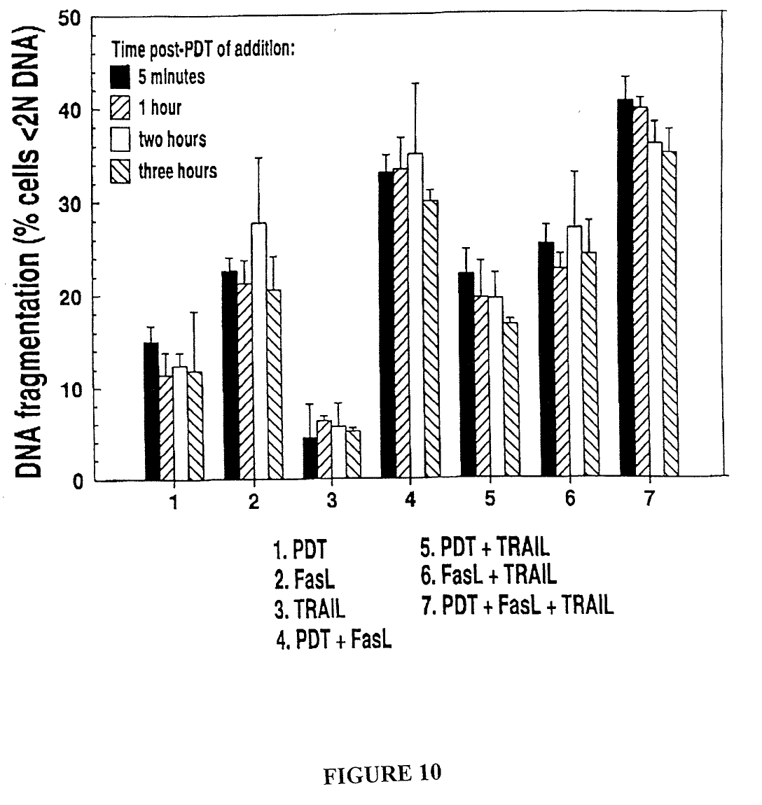

FIG. 11. Combined use of PDT and TRAIL exhibit synergistic cell killing in HeLa cells. Although over-expression of Bcl-2 or BCl-x_{L} in HeLa cells results in partial resistance to TRAIL mediated cell killing, the synergistic effect of PDT and TRAIL remains. Cell survival was determined by the MTT calorimetric assay 24 hours after treatment.

### DETAILED DESCRIPTION OF THE INVENTION

The present invention is directed to a procedure in which photodynamic therapy (PDT) is used in combination with one or more apoptosis-inducing agent(s) to enhance target cell destruction. The application of PDT in combination with apoptosis-inducing agents represents a means by which target cells that escape the immediate effects of PDT photoirradiation are still subject to destruction via apoptosis. This is especially relevant to the removal of cancer cells during anti-cancer therapy and to targeting of smooth muscle cells in therapy for intimal hyperplasia.

PDT of living cells has been found to be an apoptotic stimulus, mobilizing proteolytic (caspase) activity in such cells and leading to the degradation of specific intracellular molecules and cell death. Similar events occur in cells treated with apoptosis-inducing agents such as FasL or TRAIL. The invention is based in part upon the discovery that PDT, in combination with apoptosis-inducing agents such as TRAIL or FasL act in concert to induce more extensive levels of apoptosis. When target cells are treated with PDT at sub-optimal or sub-maximal levels, the addition of TRAIL, FasL or both TRAIL and FasL increases the extent of apoptosis in a cell population. Conversely, when target cells are exposed to apoptosis-inducing agents at sub-optimal or sub-maximal concentrations, the use of PDT in addition increases the extent of apoptosis in a cell population. As such, the application of PDT in combination with apoptosis-inducing agents permits the use of lower amounts of apoptosis-inducing agents, which is of particular benefit where higher amounts of the factors would result in deleterious effects to non-target cells. This is especially relevant in anti-cancer therapies where the selectivity for target cancer cells is desirable. Similarly, the application of apoptosis-inducing agents in combination with PDT permits the use of lower amounts of photosensitizer and/or light thereby limiting the duration of photosensitivity that is often associated with this form of therapy.

Surprisingly, the combination of PDT and apoptosis-inducing agents can result in apoptosis related effects that seem more than additive and appear synergistic. While the relationships between FasL, TRAIL and PDT induced apoptosis pathways remain not fully defined, it appears that the primary biochemical events triggered with FasL, TRAIL and PDT are distinct. Soluble recombinant Fc-Fas and Fc-TRAIL-R2 chimera proteins specifically block apoptosis with FasL and TRAIL, respectively, but do not alter the effect of PDT. This indicates that PDT-induced apoptosis proceeds independently of Fas or TRAIL-R signaling. For cells treated at a sub-optimal level of PDT, addition of FasL or TRAIL augmented caspase-3, -6 and -8 processing and activity, degradation of the caspase-3 substrate poly(ADP)polymerase (PARP) as well as further increasing the number of cells exhibiting DNA fragmentation. However, FasL or TRAIL death receptor-associated events apparently converge with PDT instigated mitochondrial events when the these treatments are applied in temporal proximity.

FasL and TRAIL mediated apoptosis proceed by distinct biochemical pathways despite their ultimate result of similar intracellular events (e.g. caspase-8 activation). Support for this view includes the distinct, and differently regulated, entities involved in FasL and TRAIL binding and receptor systems and the observation that combined application of FasL and TRAIL produces a greater level of apoptosis than either alone and the combined effect is eliminated by blocking the ability of either FasL or TRAIL to their respective receptor. Additional support is provided by the observations that cells can simultaneously express both FasL and TRAIL receptors but may be insensitive to FasL and TRAIL or sensitive to only one of these factors, which indicates a degree of separate regulation of these two pathways, and that there are distinct TRAIL “decoy” receptors, which may have counterpart FasL “decoy” receptors.

Combined use of PDT and an apoptosis-inducing agent is also of particular relevance in the treatment and prevention of vascular diseases such as atherosclerosis and other forms of intimal hyperplasia, including restenosis, transplant vascular disease, and narrowing arteriovascular fistulae. These embodiments of the invention are based on the unexpected discovery that smooth muscle cells are susceptible to TRAIL in combination with PDT. This is in contrast to the view in the art that normal cells, such as smooth muscle cells, are not susceptible to TRAIL-induced apoptosis. Without being bound by theory, it appears that PDT may generate increased oxidative stress on smooth muscle cells involved in intimal hyperplasia. Thus an additional embodiment of the invention includes the treatment of intimal hyperplasia with an apoptosis-inducing agent in the absence of PDT because smooth muscle cells are exposed to increased oxidative stress when they are involved in the formation and/or increase in arterial plaque as part of intimal hyperplasia. PDT based methods to inhibit restenosis are found in U.S. Pat. No. 5,422,362 while methods to treat arterial plaque are found in U.S. Pat. No. 5,834,503, both of which are hereby incorporated by reference in their entirety as if fully set forth. In one aspect of the invention, an appropriate photosensitizing compound is administered to a subject containing target cells in combination with an apoptosis-inducing agent. The order of administration of photosensitizer and apoptosis-inducing agent may vary, with light irradiation following administration of the photosensitizer. Simultaneous induction of apoptosis by the agent and PDT may increase the effectiveness of the methods of this invention.

After administration, the photosensitizer and apoptosis-inducing agent(s) will localize in target cells, with the photosensitizer available for photoactivation. Light of appropriate frequency and intensity will be applied using an appropriate light source, thereby activating the photosensitizer to induce apoptosis in combination with the agent(s).

As used herein, the term “target cell” refers to a living cell, including a cancer or tumor cell whether in tumors, metastases, or otherwise, a virally-infected cell, a cell involved in an autoimmune disease (such as psoriasis) or the reactions or processes thereof, cells involved in ocular neovascular disorders, cells involved in atherosclerosis, and cells involved in unwanted thrombosis and restenosis. The target cells may constitute a tissue or be part of a tissue. The target cells can be either in vitro or in vivo when targeted by the invention.

As used herein, the term “apoptosis-inducing agent” means any molecule which induces apoptosis, preferably those which bind to a cell surface receptor. These agents may be produced recombinantly, synthetically, or by isolation from naturally occurring sources. Examples of such receptors are the receptors for TRAIL and FasL noted above. Apoptosis-inducing agents include both natural and artificial ligands for the receptors, such as the TRAIL and FasL polypeptides, as well as portions and derivatives thereof, and monoclonal antibodies which bind to the TRAIL and FasL receptors. One alternative form of TRAIL for use in the invention comprises the receptor binding, C-terminal domain of TRAIL in a recombinant construct containing a leucine zipper for dimerization or multimerization (see Walczak et al. (1997) “TRAIL-R2: a novel apoptosis-mediating receptor for TRAIL.” EMBO Journal, Vol. 16, no. 17, pp. 5386-5397), are also permitted for use in the invention. Such alternative forms need only retain the ability to specifically induce apoptosis. Assays for the ability to induce apoptosis are known in the art.

The TRAIL polypeptide is described in U.S. Pat. No. 5,763,223, which is incorporated by reference. In addition, the apoptosis-inducing agents include antibodies which bind to the apoptosis-inducing receptors. Preferably, the apoptosis-inducing agents are selected from the group consisting of TRAIL or FasL or combinations thereof.

As used herein, “photosensitizer” means a chemical compound which homes to one or more types of selected target cells or tissue and, when irradiated, absorbs light to induce impairment or destruction of target cells or tissues. Photosensitizers include, but are not limited to, chlorins bacteriochlorins, phthalocyanines, porphyrins, purpurins, merocyanines, pheophorbides, and psoralens, as well as the derivatives of these compounds. Also contemplated as photosensitizers are the use of pro-drugs such as delta-aminolevulinic acid, which can produce drugs such as protoporphyrin. Additionally, expanded porphyrin-like compounds like those described in U.S. Pat. No. 5,405,957 can also be used in the methods of the invention. Preferred compounds are benzoporphyrin derivatives (BPD), monoaspartyl chlorin e6, zinc phthalocyanine, tin etiopurpurin and porfimer sodium (PHOTOFRIN®), as well as the derivatives of these compounds. Most preferred are BPD-MA, disclosed in U.S. Pat. No. 4,920,143 (which is hereby incorporated by reference as if fully set forth), B3, disclosed in U.S. patent application Ser. Nos. 08/852,494 and 09/265,245 (which are hereby incorporated by reference as if fully set forth), and EA6, disclosed in U.S. patent application Ser. Nos. 08/852,494 and 09/088,524 (which are hereby incorporated by reference as if fully set forth).

The methods and formulations of the invention generally relate to administering a photosensitizer, such as a green porphyrin, to a subject undergoing PDT in combination with administration with an apoptosis-inducing agent. Green porphyrins are in the class of compounds called benzoporphyrin derivatives (BPD). A BPD is a synthetic chlorin-like porphyrin with various structural analogues, as shown in U.S. Pat. No. 5,171,749. Preferably, the BPD is a benzoporphyrin derivative mono-acid ring A (BPD-MA), which absorbs light at about 692 nm wavelength with improved tissue penetration properties. BPD-MA, for example, is lipophilic, a potent photosensitizer, and it also appears to be phototoxic to neovascular tissues, tumors and remnant lens epithelial cells. Because of their phannokinetics, BPDs such as BPD-MA, EA6 and B3 may be the best candidates for use in the invention, but other derivatives may also be used. An optimal photosensitizer for use in the methods of the invention should be rapidly taken up by target cells and should be capable of initiating apoptosis upon irradiation with light to act in concert with the apoptosis-inducing agent.

A particularly preferred formulation according to the present invention will satisfy the following general criteria. First, an apoptosis-inducing agent capable of inducing apoptosis is utilized. Second, a photosensitizer capable of rapid entry into the target cells is used. Third, irradiation with light results in induction of apoptosis, before, after or simultaneous with the apoptosis-inducing agent mediated induction, in the target cells. These criteria do not necessarily reflect a temporal sequence of events. Conditions for exposing target cells to light after photosensitizer administration are found in U.S. Pat. Nos. 5,770,619 and 5,736,563 which are incorporated by reference.

In practice of the invention, the apoptosis-inducing agent may be administered systemically or locally, preferably systemically (Walczak et al. Nature Medicine 5: 157-163 (1999). “Tumoricidal activity of tumor necrosis factor-related apoptosis-inducing ligand in vivo.”). Moreover, the apoptosis-inducing agent may be administered before, after or simultaneous with the photosensitizer, which may also be administered to an animal either locally, for example, to the site of a tumor, or systemically. Both are described in U.S. Pat. No. 5,770,619.

After administration of the photosensitizer, sufficient time is permitted to elapse for the compound to be taken up by the target cells. This time for uptake may be varied according to various parameters, including but not limited to the photosensitizer administered, the route of administration, the physiology of the subject and of the target cells, and the artisan's skill and experience. With green porphyrins, for example, the elapsed time may be from less than about one minute to more than three hours, preferably from one minute to three hours, and more preferably from 10 to 60 minutes. The cells, or tissue containing them, then are irradiated at a wavelength of absorbence of the photosensitizer, preferably the wavelength of maximum absorbence. In the case of BPDs, the wavelength is usually between about 550 and 695 nm, as discussed above. In particular, red light is advantageous in comparison to shorter wavelength light because of its ability to penetrate more deeply into target tissue, and its relatively lower energy and the resulting lack of toxicity it poses to normal tissue while the tumor cells are destroyed.

In addition to using the invention to treat a whole animal, a population of cells or tissues may be removed from the animal, and treated in vitro. For example, this method could be used for destroying malignant or virally-infected cells from bone marrow or blood by inducing apoptosis in the cells.

Additional aspects of the invention relate to the induction of 1) apoptosis, 2) DNA fragmentation, and 3) caspase activity as well as processing, by the combined use of PDT and apoptosis-inducing agents. The induction of these individual outcomes, while being a part of the treatment and inhibition methods described above, also have applied utility in the production of DNA fragments and activated caspases as well as research utility in studies of apoptosis.

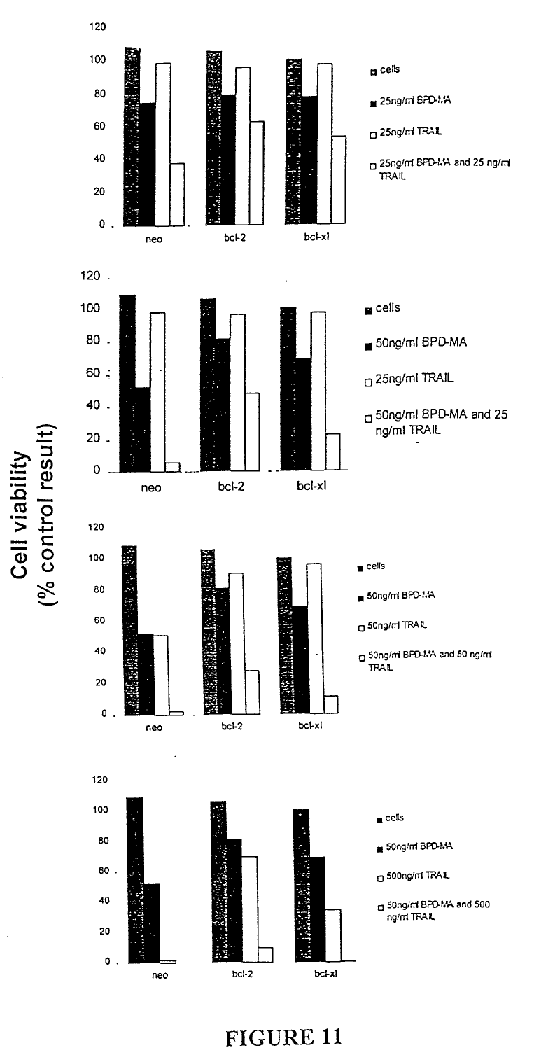

Moreover, the invention relates to the rapid and immediate induction of mitochondrial cytochrome c release by PDT, a key event in triggering apoptosis. This outcome may be utilized in the production and preparation of cytochrome c, including for industrial applications. Furthermore, the invention demonstrates an unexpected synergistic effect upon combined use of PDT and an apoptosis-inducing agent. This synergistic effect remains despite the overexpression of an inhibitor of TRAIL induced cell death. TRAIL appears to preferentially induce apoptosis in transformed, but not normal cell lines (Walczak et al. and Ashkenazi et al.). Bcl-2 is an integral membrane protein that localizes to the mitochondrial, endoplasmic reticular, and nuclear membranes (Krajewski et al.). Many proteins have been identified with Bcl-2-homologous (BH) domains. These Bcl-2 family proteins have either pro- or anti-apoptotic activities (Reed et al. and Kroemer et al.) and can influence cell survival in the face of various cytotoxic stimuli. Over-expression of Bcl-2 and Bcl-x_{L}, a distinct Bcl-2 family member, may protect against or delay the induction of apoptosis in a wide range of experimental settings (Chao et al., Kluck et al., and Yang et al.). As shown in FIG. 11, Bcl-2 or Bcl-x_{L} over-expression inhibited the loss of viability occurring following TRAIL or low-dose PDT treatment. But in combination, TRAIL and PDT produced cell death in HeLa cells despite Bcl-2 or Bcl-x_{L} over-expression in a manner that indicates synergistic action between the two treatments. The following describes the compositions and formulations of the present invention and their clinical application. Experimental data are also presented and described.

### The Photosensitizers

Photosensitizers useful in the methods of the invention include those listed above as well as the BPDs and green porphyrins, which are described in detail in Levy et al., U.S. Pat. No. 5,171,749 issued Dec. 15, 1992, and is incorporated herein by reference. “Green porphyrins” refer to porphyrin derivatives obtained by reacting a porphyrin nucleus with an alkyne in a Diels-Alder type reaction to obtain a monohydrobenzoporphyrin. Typically, green porphyrins are selected from a group of porphyrin derivatives obtained by Diels-Alder reactions of acetylene derivatives with protoporphyrin under conditions that promote reaction at only one of the two available conjugated, nonaromatic diene structures present in the protoporphyrin-IX ring system (rings A and B).

Several structures of typical green porphyrins are shown in the above cited patent, which also provides details for the production of the compounds.

Dimeric forms of the green porphyrin and dimeric or multimeric forms of green porphyrin/porphyrin combinations can be used. The dimers and oligomeric compounds of the invention can be prepared using reactions analogous to those for dimerization and oligomerization of porphyrins per se. The green porphyrins or green porphyrin/porphyrin linkages can be made directly, or porphyrins may be coupled, followed by a Diels-Alder reaction of either or both terminal porphyrins to convert them to the corresponding green porphyrins.

Additionally, the green porphyrin compounds used in the invention may be conjugated to various ligands to facilitate targeting to target cells. These ligands include those that are receptor-specific, or immunoglobulins as well as fragments thereof. Preferred ligands include antibodies in general and monoclonal antibodies, as well as immunologically reactive fragments of both.

The green porphyrin compounds of the invention may be administered as a single compound, preferably BPD-MA, or as a mixture of various green porphyrins. Suitable formulations include those appropriate for administration of therapeutic compounds in vivo. Additionally, other components may be incorporated into such formulations. These include, for example, visible dyes or various enzymes to facilitate the access of a photosensitizing compound to target cells.

### Formulations

The photosensitizers and apoptosis-inducing agents of the invention may be formulated into a variety of compositions. These compositions may also comprise further components, such as conventional delivery vehicles and excipients including isotonising agents, pH regulators, solvents, solubilizers, dyes, gelling agents and thickeners and buffers and combinations thereof. Appropriate formulations and dosages for the administration of apoptosis-inducing agents are known in the art. Suitable excipients for use with photosensitizers and apoptosis-inducing agents include water, saline, dextrose, glycerol and the like.

Typically, the photosensitizing agent is formulated by mixing it, at an appropriate temperature, e.g., at ambient temperatures, and at appropriate pHs, and the desired degree of purity, with one or more physiologically acceptable carriers, i.e., carriers that are nontoxic at the dosages and concentrations employed. Generally, the pH of the formulation depends mainly on the particular use, and concentration of photosensitizer, but preferably ranges anywhere from about 3 to about 8. Preferably, the photosensitizer is maintained at a pH in the physiological range (e.g., about 6.5 to about 7.5). The presence of salts is not necessary, and, therefore the formulation preferably is not an electrolyte solution. Appropriate nonantigenic ingredients, such as human serum albumin, may optionally be added in amounts that do not interfere with the photosensitizing agent being taken up by lens epithelial cells.

The particular concentration of a given BPD should be adjusted according to its pholosensitizing potency. For example, BPD-DA can be used but at about a five-fold higher concentration than that of BPD-MA. Moreover, the BPD may be solubilized in a different manner than by formulation in liposomes. For example, stocks of BPD-MA or any other BPD may be diluted in DMSO (dimethylsulfoxide), polyethylene glycol or any other solvent acceptable for use in the treatment of target cells.

Normally, the adjustment of pH is not required when liposomal BPD-MA is used, as both components have a neutral pH. However, when other solvents than liposomes are used, the pH may require adjustment before mixing the BPD with the other material. Since antioxidants may interfere with the treatment, they should generally should be avoided.

Preparation of dry formulations that are reconstituted immediately before use also are contemplated. The preparation of dry or lyophilized formulations of the compositions of the present invention can also be effected in a known manner, conveniently from the solutions of the invention. The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g. for some hours in a drying oven.

Suitable isotonising agents are preferably nonionic isotonising agents such as urea, glycerol, sorbitol, mannitol, aminoethanol or propylene glycol as well as ionic isotonising agents such as sodium chloride. The solutions of this invention will contain the isotonising agent, if present, in an amount sufficient to bring about the formation of an approximately isotonic solution. The expression “an approximately isotonic solution” will be taken to mean in this context a solution that has an osmolarity of about 300 milliosmol (mOsm), conveniently 300+10% mOsm. It should be borne in mind that all components of the solution contribute to the osmolarity. The nonionic isotonising agent, if present, is added in customary amounts, i.e., preferably in amounts of about 1 to about 3.5 percent by weight, preferably in amounts of about 1.5 to 3 percent by weight.

Solubilizers such as Cremophor types, preferably Cremophor RH 40, or Tween types or other customary solubilisers, may be added to the solutions of the invention in standard amounts.

A further preferred embodiment of the invention relates to a solution comprising a BPD compound, and a partially etherified cyclodextrin, the ether substituents of which are hydroxyethyl, hydroxypropyl or dihydroxypropyl groups, a nonionic isotonising agent, a buffer and an optional solvent. However, appropriate cyclodextrins should be of a size and conformation appropriate for use with the photosensitizing agents disclosed herein.

Summaries of pharmaceutical compositions suitable for use with the photosensitizers and apoptosis-inducing agents are known in the art and are found, for instance, in Remington's Pharmaceutical Sciences.

### Administration of Photosensitizers and Apoptosis-Inducing Agents

As noted above, the methods and compositions of the present invention are utilized in appropriate target cells and tissues either in an afflicted subject or in vitro. The photosensitizer and apoptosis-inducing agent containing preparations of the present invention may be administered systemically or locally and may be used alone or as components of mixtures. Preferred routes of administration are intravenous, subcutaneous, intramuscular, or intraperitoneal injections of the photosensitizers and apoptosis-inducing agents in conventional or convenient forms. In particular, liposomal or lipophilic formulations are most desirable, and injection of the apoptosis-inducing agents into target cells or tissues is one aspect of the invention. Intravenous delivery of photosensitizers is preferred, and intratissue injection may also be used when desired, as in pigmented tumor situations where the dose of PDT would be increased, for example. Oral administration of suitable oral formulations may also be appropriate in those instances where the photosensitizer may be readily administered to the target or tumor tissue via this route.

Additionally, if the treatment is to be localized to an area of metastatic tumors suitable for topical formulations, the photosensitizers may be topically administered using standard topical compositions including lotions, suspensions or pastes.

The dose of photosensitizers and apoptosis-inducing agents can be optimized by the skilled artisan depending on factors such as, but not limited to, the physical delivery system in which it is carried, the individual subject, and the judgment of the skilled practitioner. It should be noted that the various parameters used for effective PDT in the invention are interrelated. Therefore, the dose should also be adjusted with respect to other parameters, for example, fluence, irradiance, duration of the light used in PDT, and time interval between administration of the dose and the therapeutic irradiation. All of these parameters should be adjusted to produce significant damage to target cells and induce apoptosis without causing significant damage to the surrounding tissue. With photosensitizers, for example, the form of administration, such as in liposomes or when coupled to a target-specific ligand, such as an antibody or an immunologically active fragment thereof, is one factor considered by a skilled artisan.

Depending on the specificity of the preparation, smaller or larger doses of photosensitizers may be needed. For compositions which are highly specific to the target cells or tissues, such as those with the photosensitizer conjugated to a highly specific monoclonal antibody preparation or specific receptor ligand, dosages in the range of 0.05-1 mg/kg are suggested. For compositions which are less specific to the target, larger dosages, up to 1-10 mg/kg, may be desirable. The foregoing ranges are merely suggestive in that the number of variables with regard to an individual treatment regime is large and considerable deviation from these values may be expected. The skilled artisan is free to vary the foregoing concentrations so that the uptake and cellular destruction parameters are consistent with the therapeutic objectives disclosed above.

The time of apoptosis-inducing agent delivery may be before or after irradiation with light as well as before, after, or simultaneous with administration of the photosensitizer, although irradiation will occur after administration of the photosensitizer. The apoptosis-inducing agents may be delivered immediately after irradiation. This may be of particular relevance with apoptosis-inducing agents that are opaque or otherwise interfere with irradiation.

Without being bound by theory and in instances of BPDs being used as the photosensitizer, irradiation is thought to result in the interaction of BPD in its triplet state with oxygen and other compounds to form reactive intermediates, such as singlet oxygen, which can cause disruption of cellular structures. Possible cellular targets include the cell membrane, mitochondria, lysosomal membranes.

Each photosensitizer requires activation with an appropriate wavelength of light. With BPDs, an appropriate light source, preferably a laser or laser diode, in the range of about 550 to about 695 nm, is used to destroy target cells. An appropriate and preferred wavelength for such a laser would be 690±12.5 nm at half maximum. Cell destruction may commence in as little as 60 seconds, and likely is sufficiently begun within about 15 to about 30 seconds. The light dose administered during the PDT treatment contemplated herein can vary, but preferably ranges between about 10 to about 150 J/cm². The range between about 50-100 J/cm² is preferred. Increasing irradiance may decrease the exposure times.

Localized delivery of light is preferred, and delivery localized to the target is more preferred. Delivery of light prior to photosensitizer activating light is also contemplated to improve penetration of the activating light. For example, irradiation of pigmented melanomas with infrared light before visible red light bleaches the melanin to improve penetration of the red light.

The time of light irradiation after administration of the photosensitizer may be important as one way of maximizing the selectivity of the treatment, thus minimizing damage to structures other than the target cells and tissues. Light treatment immediately, or shortly, after administration of the photosensitizer should generally be attempted.

The following examples are intended to illustrate but not to limit the invention.

### EXAMPLE 1

### Methods Used

### In General

The capacity of PDT to act in combination with FasL and/or TRAIL, factors known to cause apoptosis by signaling through specific cell surface receptors, against human Jurkat T cell lymphoma cells was evaluated by a number of different criteria. These included

The presence of hypodiploid levels of DNA, an indicator of cells exhibiting DNA fragmentation during apoptosis;

Determination of the activity levels of caspase-3, -6 and -8 by means of protease assays performed with whole cell extracts; and/or

Assessment of the state of caspase-3, -6, -8 and -9 and the caspase substrate PARP by Western immunoblot analyses.

### Photodynamic Treatment of Cells

The human Jurkat T cell line containing a neomycin resistance gene was obtained from Dr. Charles Rudin (University of Chicago). Cells were maintained in RPMI 1640 medium containing 10% heat-inactivated fetal calf serum (FCS) with penicillin (100 U/mL), streptomycin (100 μg/mL), 20 mM HEPES, 2 mM L-glutamine and G418 (1 μg/ml) purchased from Gibco BRL (Burlington, Ontario) in a CO₂ incubator at 37° C. To elicit apoptosis with PDT, Jurkat cells were incubated with different amounts of liposomally formulated benzoporphyrin derivative monoacid ring A (BPD-MA, verteporfin, QLT PhotoTherapeutics, Inc., Vancouver, Canada) for one hour at 37° C. under light-protected conditions. The culture medium used for experiments was identical to that used for the passage of the cells except that FCS was used at a concentration of 5%. Experiments were conducted either in 6-well culture plates at 5×10⁶ cells in a volume of 4 ml per well or in 96-well microtiter plates at 1×10⁵ cells per well at 0.2 ml per well. Cells were irradiated with light of a wavelength of 690 nm delivered at 65-73 mW/sec from light emitting diodes (LED) to achieve a total light dose of 5 J/cm². Cells were returned to the incubator until required for analysis.

### Treatment of Cells with FasL and TRAIL

FasL and TRAIL as used were recombinant preparations. FasL was obtained from Upstate Biotechnology (Lake Placid NY) and corresponded to amino acids 103-281 of the soluble domain of human FasL. TRAIL was obtained from BIOMOL Research Laboratories (Plymouth Meeting, Pa.) and corresponded to residues 114-281 of the soluble domain of human TRAIL. FasL and TRAIL were usually added immediately following the treatment of cells with PDT, although in one series of experiments these factors were added at 1, 2 or 3 hours post-irradiation.

### Flow Cytometric Detection of Apoptosis

The proportion of cells containing sub-diploid levels of DNA 24 hours following treatment was determined using a propidium iodide (“PI”) fluorescence analysis procedure as described by Telford et al. (“Rapid Quantitation of Apoptosis in Pure and Heterogeneous Cell Populations Using Flow Cytometry” J. Immunol Methods 172:1-6 (1994)) or by Darzynkieicz et al. (“Features of Apoptotic Cells Measured by Flow Cytometry”, Cytometry, 13:795-808 (1992)).

Twenty four hours following PDT, 5×10⁵ cells were washed twice with ice-cold PBS and then permeablized and fixed in 80% ethanol at 4° C. for one hour. The cells were again washed in ice-cold PBS and stained with PI (50 μg/mL) in PBS with simultaneous deoxyribose nuclease-free ribonuclease A (5 U/mL) treatment. These samples were analyzed by flow cytometry. The percentage of apoptotic cells was calculated using single color analysis for PI fluorescence with a Coulter XL flow cytometer (Coulter Electronics, Inc., Hialeah, Fla.). Cells undergoing apoptosis exhibit DNA fragmentation and therefore have fewer available sites for PI intercalation, leading to lower levels of fluorescence for these cells. The degree of separation between the apoptotic population and the G₀/G₁ peak is readily apparent and the percentage of apoptotic cells is determined from the subsequent histogram.

### Preparation of Cell Extracts

Whole cell lysates were prepared by washing 1×10⁷ cells per sample twice with ice-cold PBS. Cells were disrupted with 1 mL of lysis buffer [1% Nonidet P-40 detergent (NP-40), 20 mM Tris-HCl, pH 8.0, 137 mM NaCl, 10% glycerol, 1 mM phenylmethylsulfonyl fluoride (PMSF) aprotinln (0.15 U/mL) and 1 mM sodium orthovanadate] for 20 minutes on ice. Lysates were centrifuged for 10 minutes at 15,800×g at 4° C.

### Protease Assays

Different caspases exhibit proteolytic activity at specific amino acid sequences of proteins. These sequences are denoted by letter codes for each amino acid. To evaluate relative DEVDase (caspases 3 and 7), VEIDase (caspase 6) and IETDase (caspase 8) cleavage activity, Jurkat cell lysates were incubated with caspase-specific fluorescent substrates as described by Granville et al. (“Photodynamic therapy induces caspase-3 activation in HL-60 cells”, Cell Death Differ., 4, 623-629 (1997)). Briefly, lysates were incubated with reaction buffer (20 mM Tris pH 7.5, 137 mM NaCl, 1% NP-40, 10% glycerol) containing 100 μM of the caspase substrates Ac-DEVD-AMC (Calbiochem, Cambridge, Mass.), Ac-VEID-AMC (Calbiochem, Cambridge, Mass.), Ac-lETD-AMC (BIOMOL, Plymouth Meeting Pa.), and Ac-LEHD-AMC (Calbiochem, Cambridge, Mass.). The letter codes for the amino acid residues present within the inhibitor peptides are as following: D=aspartic acid; E=glutamic acid; H=histidine; I=isoleucine; T=threonine; V=valine. The reaction mixture was incubated at 37° C. for 1 (DEVD.AMC) or 24 (VEID.AMC and IETH.AMC) h and fluorescence was measured using an excitation at 380 nm and an emission wavelength of 460 nm with a CytoFluor™ 2350 fluorescent measuring system (Perseptive Biosystems, Burlington, Ont).

### Preparation of Cytosolic Extracts

To obtain cytosolic extracts, cells were treated with 0.6 ml of ice-cold buffer [250 mM sucrose, 20 mM Hepes pH 7.4, 10 mM KCl, 1.5 mM MgCl₂, 1 mM EGTA, 1 mM EDTA, 1 mM dithiothreitol, 1 mM PMSF and aprotinin (10 μg/ml)]. Cells were disrupted by 20 strokes with the B pestle of a Kontes™ dounce homogenizer. Supernatant collected following centrifugation at 10000×g was further centrifuged at 100000×g for 1 h at 4° C. in a Beckman Optima™ ultracentrifuge using a TL-100 rotor. (Granville et al., “Rapid cytochrome c release, activation of caspases 3, 6, 7 and 8 followed by Bap31 cleavage in HeLa cells treated with photodynamic therapy”, FEBS Lett. 437, 5-10 (1998)).

### Western Immunoblot Analysis

To evaluate the activation state of different caspases and the status of other cellular proteins, whole cell lysates were separated by sodium dodecyl sulphate polyacrylamide gel electrophoresis (SDS-PAGE). Specifically, detergent-soluble proteins were separated by SDS-PAGE within 10% gels under reducing conditions as described by Laemmli et al. (“Cleavage of Structural Proteins During the Assembly of the Head of Bacteriophage T4”, Nature, 227, 680-685 (1970)). The proteins were transferred to nitrocellulose membranes at 100 volts for one hour. These membranes were treated for 30 minutes at room temperature with 5% skim milk powder in PBS to block “non-specific” sites. The blocked membranes were incubated for 45 minutes using the following antibodies and concentrations:

Mouse IgG1 monoclonal anti-caspase-3 antibody (clone number E-8; catalog number sc-7272; 1 μg/ml) was from Santa Cruz Biotechnology (Santa Cruz Calif.).

Mouse IgG2b monoclonal antibody against denatured cytochrome c (clone number 7H8.2C12; catalog number 65981A; 1 μg/ml) was from PharMingen (San Diego Calif.).

Polyclonal rabbit anti-caspase-9 (catalog number 68086; dilution of 1:3333) was purchased from PharMingen (San Diego, Calif.).

Polyclonal rabbit IgG anti-caspase-6 antibody (catalog number 06-691; 1 μg/ml) was from Upstate Biotechnology, Inc. (Lake Placid, N.Y.).

Mouse IgG2b monoclonal anti-caspase-8 antibody (clone number 5F7; catalog number 05-477; 2 μg/ml) was from Upstate Biotechnology, Inc. (Lake Placid, N.Y.).

Monoclonal antibody against poly(ADP-ribose) polymerase (PARP) (clone C-2-10; catalog number SA-249; (0.24 μg/ml) was from BIOMOL (Plymouth Meeting, Pa.). Following incubation with the primary antibody, membranes were washed with PBS containing 0.05% Tween 20 and then probed with either horseradish peroxidase (HRP)-conjugated anti-mouse or anti-rabbit IgG antibodies from Transduction Laboratories (Lexington, Ky.) at 1:3333 dilutions. Proteins were detected using the enhanced chemiluminescent detection system (Amersham, Canada) and bands visualized by autoradiographic techniques. Gels were viewed with a HP ScanJet 4c (Hewlett Packard, Palo Alto, Calif.) and band densities were measured using ID Image Analysis Software (Eastman Kodak Company, Rochester, N.Y.).

### EXAMPLE 2

### DNA Fragmentation by PDT and Apoptosis-Inducing Agents

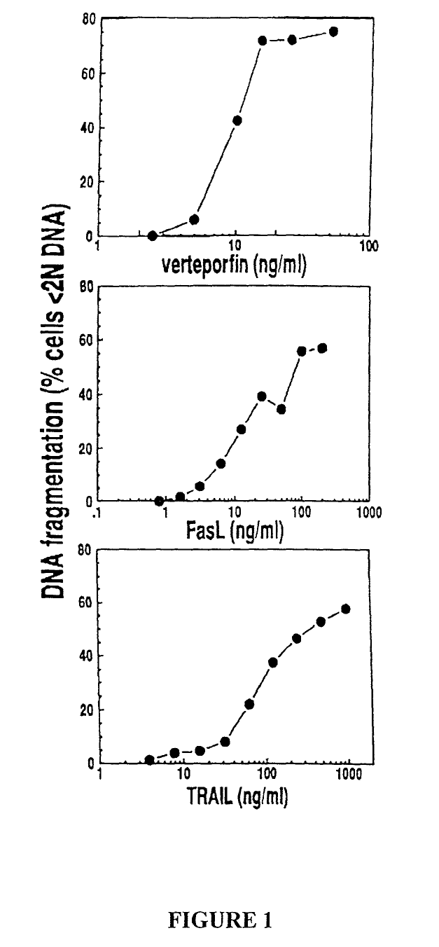

For Jurkat cells treated with different amounts of verteporfin and a constant amount of red light, a drug dose-related increase in the level of DNA fragmentation was evident upon analysis 24 hours later (FIG. 1). A relatively steep response curve was apparent in the impact of PDT upon DNA fragmentation. In this experiment, verteporfin at 5 ng/ml produced DNA fragmentation in a minor proportion of cells while at 15 ng/ml the vast majority of cells exhibited sub-diploid DNA levels following light irradiation. In contrast, more gradual DNA fragmentation response curves were evident for Jurkat cells treated with increasing amounts of FasL or TRAIL. FasL at 200 ng/ml and TRAIL at 1000 ng/ml induced DNA fragmentation in approximately 60% of the cells within 24 hours.

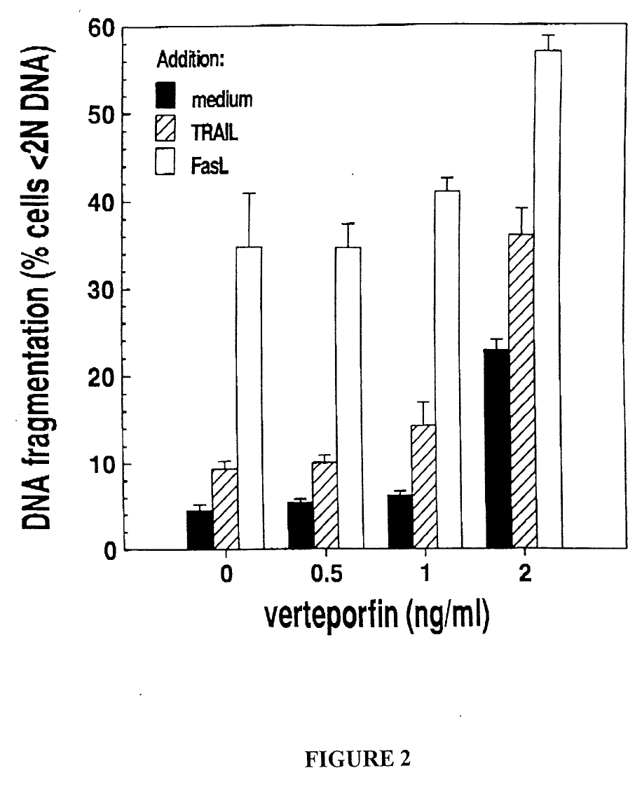

The impact of PDT combined with TRAIL or FasL on Jurkat cell apoptosis was then examined. At the concentrations employed, TRAIL by itself produced approximately a two-fold increase in the number of cells displaying DNA fragmentation while FasL produced about a seven-fold increase in this parameter (FIG. 2). When the same amount of TRAIL or FasL was added to cells treated with verteporfin (0.5 and 1 ng/ml) and a constant amount of red light, a greater proportion of cells contained a sub-diploid level of DNA than cells treated with PDT alone. Five times more light-irradiated cells treated with verteporfin at 2 ng/ml exhibited DNA fragmentation than cells treated with light alone. Addition of recombinant FasL and TRAIL to cells treated with verteporfin at 2 ng/ml increased the proportion of cells containing a sub-diploid amount of DNA by approximately eight-fold with TRAIL and twelve-fold with FasL in comparison to the levels of light-treated control cells. Surprisingly, the combination of PDT and TRAIL produced a greater than additive result: compare 2 ng/ml verteporfin with TRAIL to 2 ng/ml verteporfin in the absence of TRAIL and TRAIL alone (0 ng/ml verteporfin).

As such, PDT with verteporfin, as well as FasL and TRAIL treatment resulted in DNA fragmentation and the induction of apoptosis. Moreover, the combination of PDT with these apoptosis-inducing agents resulted in increased levels of DNA fragmentation.

### EXAMPLE 3

### Induction of Caspase Processing and Activity by PDT and the Apoptosis-Inducing Agent FasL

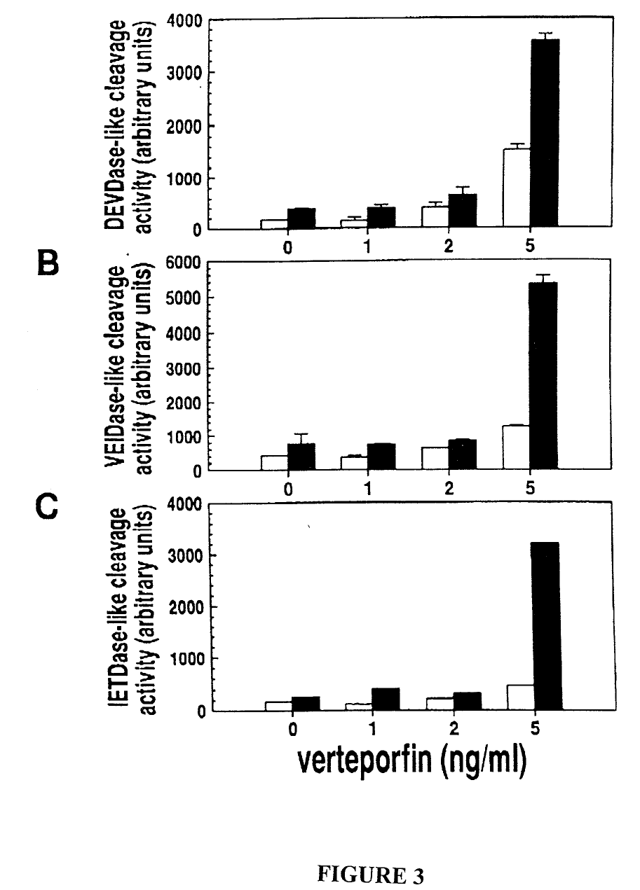

The impact of FasL combined with PDT upon cellular caspase activity levels was tested. Exposure of Jurkat cells to FasL for 3 hours increased DEVDase-like (FIG. 3A) and VEIDase-like (FIG. 3B) cleavage activity by approximately two-fold. Addition of FasL increased IETDase-like activity by one-and-half fold (FIG. 3C) within whole cell extracts as compared to the levels for control cells. These observations demonstrated the mobilization of caspase-3, -6 and 8, respectively, in the apoptotic response to FasL. Treatment of Jurkat cells with verteporfin and light, in the absence of FasL addition, also promoted DEVDase-like, VEIDase-like and IETDase-like activity within cell extracts as determined by the analysis of cell extracts at 3 hours post-PDT. The effect of PDT on caspase activity was greatest at a verteporfin concentration of 5 ng/ml. For extracts prepared from cells treated with FasL as well as PDT, caspase cleavage activity levels were 2-5 fold higher at a verteporfin concentration of 5 ng/ml than when PDT or FasL were applied independently. This reflects a greater than additive effect between PDT and FasL treatments.

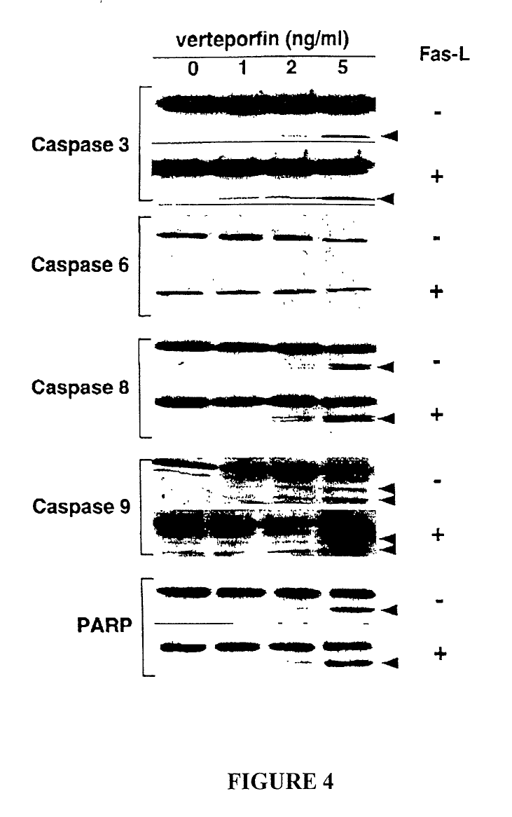

Evaluation of the status of different caspases by immunoblot analysis supported the protease assay results for the combined effects of PDT and FasL. By 3 hours following light irradiation, cells incubated with verteporfin exhibited evidence of caspase-3, -6, -8 and -9 processing with increasing verteporfin concentrations (FIG. 4). At this sampling time, little or no processing of these caspases or PARP was noted for cells exposed to FasL. However, for cells treated with PDT and the same amount of FasL, more extensive caspase processing and PARP degradation was detectable than with PDT or FasL alone. Since the antibody used to detect caspase-6 did not recognize the activated form of this caspase, processing of this protease was discernible by a decrease in the band intensity of the proform. The observed changes in band densities were confirmed by densitometric analyses.

As such, PDT with verteporfin, as well as FasL treatment resulted in caspase activation and the processing of a caspase target. Moreover, the combination of PDT with FasL resulted in increased levels of activation.

### EXAMPLE 4

### Induction of Caspase Processing and Activity by PDT and the Apoptosis-Inducing Agent TRAIL

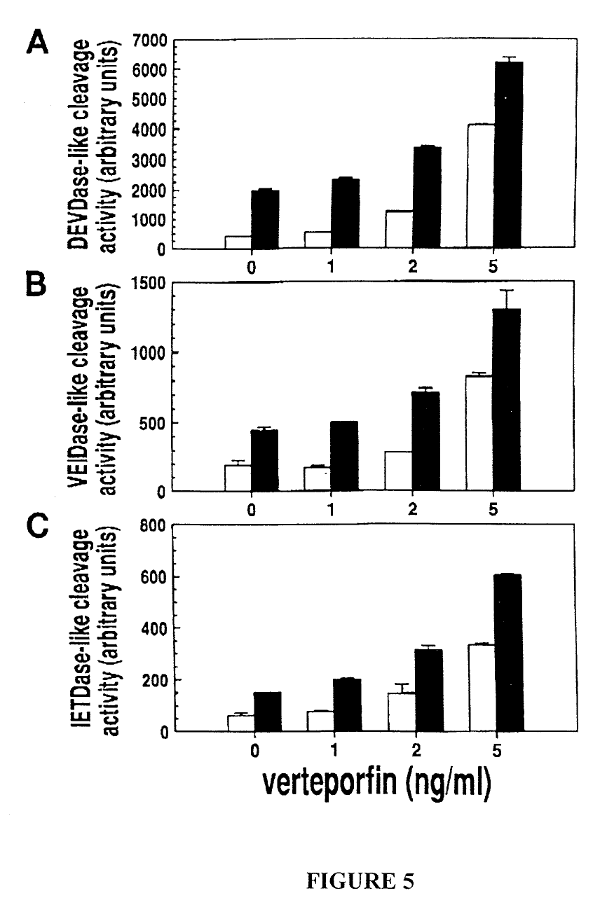

Exposure of Jurkat cells to TRAIL for 3 hours elevated DEVDase-like activity by approximately four-fold (FIG. 5A), VEIDase-like activity by two-fold (FIG. 5B) and IETDase-like activity by two-fold (FIG. 5C) within whole cell extracts as compared to the levels for control cells. These observations suggested a mobilization of caspase-3, -6 and -8, respectively, in the apoptotic response to TRAIL. Treatment of Jurkat cells with verteporfin and light, in the absence of TRAIL addition, also lead to increased levels of DEVDase-like, VEIDase-like and IETDase-like activity within cell extracts prepared at 3 hours post-PDT. This effect of PDT was most marked at verteporfin concentration of 5 ng/ml. For extracts prepared from cells treated with TRAIL as well as PDT, levels of caspase activity were higher for each verteporfin concentration than when either PDT or TRAIL was applied independently.

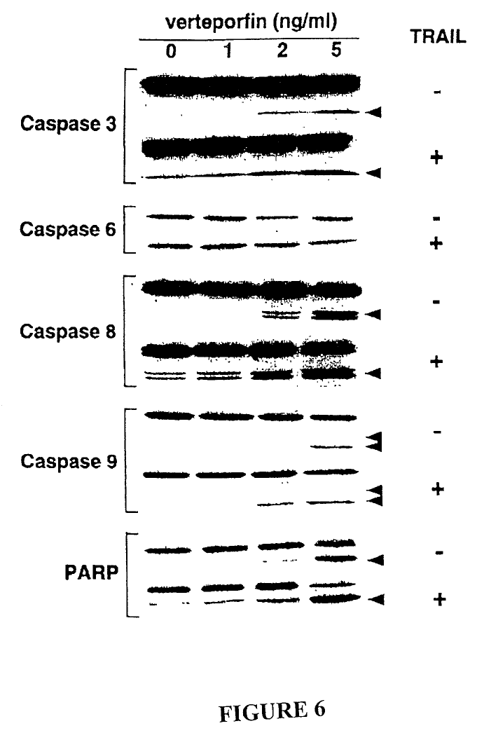

Evaluation of caspase status by immunoblot analysis supported protease assay results. By 3 hours following light irradiation, cells exhibited processing of caspases-3, -6, -8 and -9 with increasing verteporfin concentrations (FIG. 6). At this sampling time, minor processing of these caspases and PARP was noted for cells exposed to TRAIL alone. However, for cells treated PDT and the same amount of TRAIL, more extensive caspase processing and PARP degradation was detectable than with PDT or TRAIL alone. These observed changes in band densities were confirmed by densitometric analyses.

As such, PDT with verteporfin, as well as TRAIL treatment resulted in caspase activation and the processing of a caspase target. Moreover, the combination of PDT with TRAIL resulted in increased levels of activation.

### EXAMPLE 5

### Induction of Caspase Processing and Activity by PDT and Apoptosis-Inducing Agents

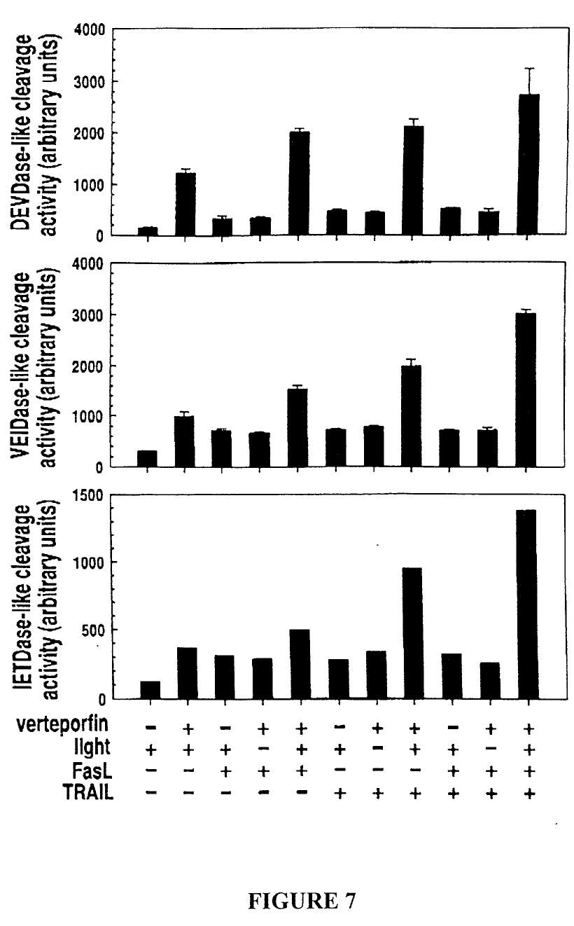

FasL and/or TRAIL in combination with PDT upon caspase activity was also tested (FIG. 7). As anticipated, PDT, FasL and TRAIL modestly increased Jurkat cell protease activity as evidenced by the processing of fluorogenic DEVD, VEID and IETD peptides. Combinations of FasL and PDT or TRAIL and PDT yielded higher caspase activity than when these treatments were given independently. In the absence of light irradiation, the level of protease activity observed following the exposure of Jurkat cells to verteporfin and FasL or TRAIL was no different than that produced by FasL or TRAIL alone. Together FasL, TRAIL and PDT elicited the greatest level of protease activity. However, this combined effect of verteporfin, FasL and TRAIL upon protease activity was not observed if the cultures were protected from light.

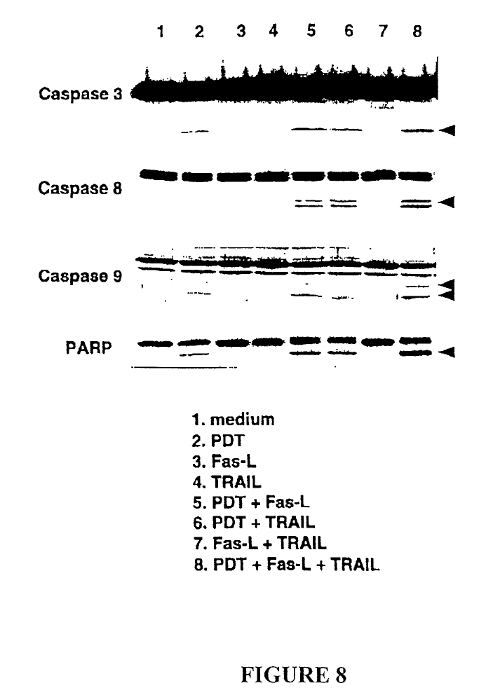

Immunoblot analyses were performed to evaluate the status of caspases-3, -8, and -9 as well as PARP for Jurkat cells treated with PDT combined with FasL and/or TRAIL. Extracts prepared from cells incubated with verteporfin and light-irradiated 3 hours before displayed a moderate degree of caspases-3, -8, -9 and PARP.processing (FIG. 8). For cells treated in parallel with FasL or TRAIL alone there was little detectable processing of these proteins. However, combinations of PDT and FasL as well as PDT and TRAIL lead to a higher degree of processing of these proteins than produced by PDT, FasL or TRAIL separately. Furthermore, in combination, PDT, FasL and TRAIL lead to the most extensive level of processing of these caspases and PARP as indicated by the increased band density of their respective cleavage products. The observed changes in band densities were confirmed by densitometric analyses.

Again, PDT with verteporfin, in combination FasL, TRAIL, or both, resulted in increased levels of caspase activation and the processing of a caspase target.

### EXAMPLE 6

### Induction of Cytochrome c

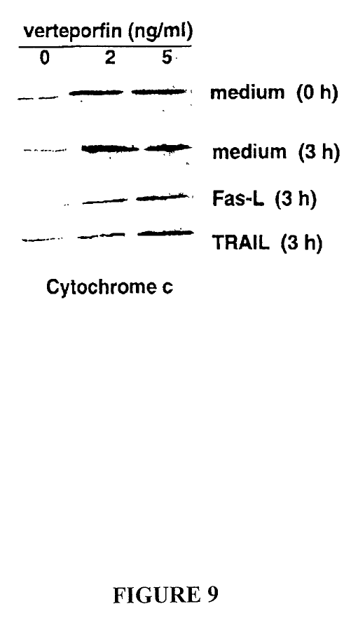

Treatment of Jurkat cells with verteporfin and light produced the immediate appearance of cytochrome c within the cytosolic fraction (FIG. 9A). Cytosolic fractions prepared immediately following and three hours after photoirradiation contained comparable levels of cytochrome c. Cytosolic extracts prepared from cells treated with FasL or TRAIL 3 hours before contained background levels of cytochrome c (FIG. 9B). However, cytosolic extracts of cells treated with PDT in combination with TRAIL or FasL contained readily detectable amounts of cytochrome c.

Clearly, PDT resulted in the immediate appearance of cytochrome c within the cytosol fraction while neither FasL or TRAIL, at the concentrations employed, exhibited this activity.

### EXAMPLE 7

### Augmentation of PDT Induced DNA Fragmentation by Apoptosis-Inducing Agents

TRAIL and/or FasL were added at increasing times in order to assess their ability to augment apoptosis in PDT-treated cells. Jurkat cells were initially treated with light alone or verteporfin and light. TRAIL and/or FasL were added approximately 5 minutes or 1, 2 or 3 hours after PDT. DNA fragmentation levels were determined 24 hours after PDT. For cells from all treatment times, DNA fragmentation was detectable for cells treated with verteporfin and light as well as those incubated with FasL and/or TRAIL. The capacity of FasL and/or TRAIL to accentuate PDT-mediated apoptosis in Jurkat cells was still demonstrable even when these recombinant factors were added up to 3 hours after PDT. However, a modest decline in this combined effect of PDT with FasL and/or TRAIL was observed when the recombinant factors were added at 3 hours post-PDT. FasL and TRAIL individually or in combination induced a comparable level of DNA fragmentation when they were added to the cells at all time points.

Again, PDT with verteporfin, in combination FasL, TRAIL, or both, resulted in increased levels of DNA fragmentation and induction of apoptosis.

### EXAMPLE 8

### Combined Use of PDT and an Apoptosis-Inducing Agent Exhibits Synergistic Effects in Cell Death

HeLa cells were from the American Type Culture Collection (Manassas, Va.). Cells were grown in Dulbecco's modified Eagles medium (DMEM) supplemented with penicillin (100 U/ml), streptomycin (100 μg/ml), 2 mM L-glutamine, 1 mM HEPES buffer, and 10% heat-inactivated fetal bovine serum (FBS). HeLa cells over-expressing Bcl-2 and Bcl-x_{L} were generated as described (Vander Heiden et al.). Briefly, Bcl-2 and Bcl-x_{L} inserts were cloned into an EcoRI site of a pSFFV-neo vector (Fuhlbrigge et al.). Vectors containing inserts, or no insert (neo), were transfected into HeLa cells by electroporation. After selection in G418 (Gibco/BRL Life Technologies), cells were cloned by limiting dilution and transfectants screened for Bcl-x_{L} or Bcl-2 by Western blot analysis. Transfected cell lines were maintained in complete DMEM with G418 (10 μg/ml).

For treatments with TRAIL and PDT, cells were incubated with 0-100 ng/ml verteporfin for 60 min at 37° C. in complete DMEM with 2% FBS or incubated with medium alone. Cells were light protected or exposed to blue fluorescent light to give a total dose of 1 J/cm². For experiments where cells were treated with 0-1000 ng/ml TRAIL (BIOMOL, Plymouth Meeting, Pa.), this compound was added immediately after light treatment to HeLa cells in complete DMEM with 10% FBS. Cells were maintained at 37° C. with 5% CO₂

To assess their viability, cells were cultured in 96-well microtiter plates and the MTT (3-(4,5)-dimethylthiazol-2-yl)-2,5-diphenyl tetrazolium bromide) calorimetric assay was performed as described (Mosmann et al.) with modifications. Following treatment, culture medium was decanted and replaced with complete DMEM containing 10% FBS. At 24 h following treatment, culture medium was aspirated and 50 μg of MTT in 100 μl of DMEM containing 2% FBS was added to each well. The reaction was stopped 2 h later by the addition of 150 μl of acidified isopropanol. Color development was measured at 590 nm with a microtiter plate reader. The result obtained for PDT, TRAIL or TRAIL plus PDT treatments was compared to the MTT value obtained for cells not given any treatment and expressed as percentages (%) of the control result.

HeLa/neo, HeLa/Bcl-2, and HeLa/Bcl-x_{L} cells were evaluated for suscptibility to PDT and TRAIL-mediated killing. Cell viability was determined 24 hours post-treatment by MTT dye reduction assay. There was little or no loss of cell viability in HeLa/neo, HeLa/Bcl-2, or HeLa/Bcl-x_{L} treated with TRAIL up to 25 ng/ml. For HeLa/neo, but not HeLa/Bcl-2 or HeLa/Bcl-x_{L}, cells treated with TRAIL at 50 and 100 ng/ml there was a loss in viability. At 500 ng/ml, TRAIL produced a complete loss of viability for the HeLa/neo cells while a significant degree of protection against TRAIL was evident for HeLa/Bcl-2 and Bcl-x₁/HeLa cells. PDT with verteporfin at 25 or 50 ng/ml resulted in some loss in viability for all three cell types. PDT in combination with TRAIL significantly decreased cell viability beyond the result for either PDT or TRAIL alone and beyond the expected effect if the two treatments acted together in an additive fashion.

Thus for cells that may have developed resistance to TRAIL alone based on Bcl expression, combined treatment with PDT and TRAIL remains a viable alternative therapy.

### References

1. Wiley S R, Schooley K, Smolak P J, Din W S, Huang C P, Nicholl J K, et al. Identification and characterization of a new member of the TNF family that induces apoptosis. Immunity (1995) 3: 673-682.

2. Pitti R M, Marsters S A, Ruppert S, Donahue C J, Moore A, Ashkenazi A. Induction of apoptosis by Apo-2 ligand, a new member of the tumor necrosis factor cytokine family. J Biol Chem (1996) 271: 12687-90.

3. Walczak H, Miller R E, Ariail K, Gliniak B, Griffith T S, Kubin M, et al. Tumoricidal activity of tumor necrosis factor-related apoptosis-inducing ligand in vivo. Nat Med (1999) 5:157-63.

4. Ashkenazi A, Pai R C, Fong S, Leung S, Lawrence D A, Marsters S A, et al. Safety and antitumor activity of recombinant soluble Apo2 ligand. J Clin Invest (1999)104:155-62.

5. Krajewski S, Tanaka S, Takayama S, Schibler M J, Fenton W, Reed J C. Investigation of the subcellular distribution of the bcl-2 oncoprotein: residence in the nuclear envelope, endoplasmic reticulum, and outer mitochondrial membranes. Cancer Res. (1993) 53:4701-4714.

6. Reed J C. Double identity for proteins of the Bcl-2 family. Nature (1997) 387:773-776.

7. Kroemer G. The proto-oncogene Bcl-2 and its role in regulating apoptosis. Nature Med. (1997) 3:614-620.

8. Chao D T, Linette G P, Boise L H, White L S, Thompson C B, Korsemeyer S J. BcI-x_{L} and Bcl-2 repress a common pathway of cell death. J Exp Med (1995) 182:821-828.

9. Kluck R M, Bossy-Wetzel E, Green D R, Newmeyer D D. The release of cytochrome c from mitochondria: a primary site for Bcl-2 regulation of apoptosis. Science (1997) 275:1132-1136.

10. Yang J, Liu X, Bhalla K, Kim C N, Ibrado A M, Cai J, et al. Prevention of apoptosis by Bcl-2: release of cytochrome c from mitochondria blocked. Science (1997) 275:1129-1132.

11. Vander Heiden M G, Chandel N S, Williamson E K, Schumacker P T, Thompson C B. Bcl-xL regulates the membrane potential and volume homeostasis of mitochondria. Cell (1997) 91:627-37.

12. Fuhlbrigge R C, Fine S M, Unanue E R, Chaplin D D. Expression of membrane interleukin 1 by fibroblasts transfected with murine pro-interleukin 1α cDNA. Proc Natl Acad Sci U S A (1988) 85:5649-53.

13. Mosmann T. Rapid colorimetric assay for cellular growth and survival: application to proliferation and cytotoxicity assays. J Immunol Methods (1983) 57:55-60.

All references cited herein are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth as follows in the scope of the appended claims.

We claim:

1. A method of enhancing apoptosis mediated destruction of target cells, comprising
(a) exposing the target cells to an amount of a photosensitizer sufficient to permit an effective amount to localize in the cells;
(b) permitting sufficient time to elapse to allow an effective amount of the photosensitizer to localize in the target cells; and
(c) irradiating the target cells with light absorbed by the photosensitizer at an sufficient energy level to induce apoptosis; and
(d) contacting said cells with an apoptosis-inducing agent.

2. A method of enhancing the destruction of target cells, which method comprises
contacting said target cells with an apoptosis-inducing agent after photodynamic therapy (PDT) of said cells to induce apoptosis mediated target cell destruction.

3. A method of activating caspase activity in cells comprising
contacting said cells with an apoptosis-inducing agent after PDT of said cells, resulting in induction of caspase activity.

4. The method of claim 1 wherein the PDT comprises the use of a green porphyrin as the photosensitizer.

5. The method of claim 1 wherein said apoptosis-inducing agent is selected from FasL, TRAIL, recombinant forms of FasL or TRAIL, or any combination thereof.

6. The method of claim 5 wherein said apoptosis-inducing agent is FasL or a recombinant form thereof which retains apoptosis inducing activity.

7. The method of claim 5 wherein said apoptosis-inducing agent is TRAIL or a recombinant form thereof which retains apoptosis inducing activity.

8. The method of claim 7 wherein said apoptosis-inducing agent is a recombinant form of TRAIL comprising the C-terminal receptor binding domain and a leucine zipper.

9. The method of claim 2 wherein said PDT comprises exposure to photosensitizer and said contacting with apoptosis-inducing agent is by intravenous or systemic administration.

10. The method of claim 1 wherein said PDT comprises exposure to photosensitizer by localized administration.

11. The method of claim 10 wherein said PDT comprises irradiation localized to the target cells.

12. The method of claim 1 wherein said PDT comprises irradiation localized to the target cells.

13. The method of claim 1 wherein said target cells are tumor cells or smooth muscle cells.

14. The method of claim 2 wherein said target cells are tumor cells or smooth muscle cells.

15. The method of claim 9 wherein said photosensitizer is a green porphyrin.

16. The method of claim 9 wherein said photosensitizer is administered locally.

17. The method of claim 9 wherein said PDT comprises irradiation localized to the target cells.

18. The method of claim 2 wherein said apoptosis-inducing agent is selected from FasL, TRAIL, recombinant forms of FasL or TRAIL, or any combination thereof.

19. The method of claim 3 wherein said apoptosis-inducing agent is selected from FasL, TRAIL, recombinant forms of FasL or TRAIL, or any combination thereof.

20. The method of claim 3 wherein the PDT comprises the use of a green porphyrin as the photosensitizer.
